(19) United States
(12) Patent Application Publication
(10) Pub. No.: US 2019/0382739 A1
(43) Pub. Date: Dec. 19, 2019

(54) MODIFIED YEAST CELLS THAT OVEREXPRESS A DNA POLYMERASE SUBUNIT

(71) Applicant: DANISCO US INC., Palo Alto, CA (US)

(72) Inventors: Celia Emily Gaby Payen, Palo Alto, CA (US); Min Qi, Palo Alto, CA (US)

(21) Appl. No.: 16/478,912

(22) PCT Filed: Jan. 16, 2018
(86) PCT No.: PCT/US18/13776

§ 371 Date: Jul. 18, 2019

Related U.S. Application Data
(60) Provisional application No. 62/447,845, filed on Jan. 18, 2017.

(51) Int. Cl. C12N 9/12; C12P 7/06; C12P 7/16; C12N 1/16; C12N 15/67

(52) U.S. Cl. CPC C12N 9/1252; C12P 7/06; C12Y 207/07007; C12N 1/16; C12N 15/67; C12P 7/16

(57) ABSTRACT

Described are compositions and methods relating to modified yeast cells that overexpress a DNA polymerase II subunit, resulting in increased alcohol production. Such yeast cells are well-suited for use in commercial fuel alcohol production to increase yield.

### CROSS REFERENCE TO RELATED APPLICATIONS

This application claims benefit to U.S. Provisional Application No. 62/447,845, filed Jan. 18, 2017, which is hereby incorporated by reference in its entirety.

### TECHNICAL FIELD

The present compositions and methods relate to engineered yeast that overexpress a DNA polymerase II subunit resulting in increased alcohol production from a starch-containing substrate. Such yeast is well-suited for use in fuel alcohol production to increase yield.

### BACKGROUND

Many countries make fuel alcohol from fermentable substrates, such as corn starch, sugar cane, cassava, and molasses. According to the Renewable Fuel Association (Washington D.C., United States), 2015 fuel ethanol production was close to 15 billion gallons in the United States, alone.

Butanol is an important industrial chemical and drop-in fuel component with a variety of applications including use as a renewable fuel additive, a feedstock chemical in the plastics industry, and a food-grade extractant in the food and flavor industry. Accordingly, there is a high demand for alcohols such as butanol and isobutanol, as well as for efficient and environmentally-friendly production methods.

In view of the large amount of alcohol produced in the world, even a minor increase in the efficiency of a fermenting organism can result in a tremendous increase in the amount of available alcohol. Accordingly, the need exists for organisms that are more efficient at producing alcohol.

### SUMMARY

Described are compositions and methods relating to modified yeast cells that overexpress a DNA polymerase II subunit resulting in increased alcohol production from a starch-containing substrate, and methods of use, thereof. Aspects and embodiments of the modified yeast cells and methods are described in the following, independently-numbered paragraphs.

1. In one aspect, modified yeast cells derived from parental yeast cells are provided, the modified cells comprising a genetic alteration that causes the modified cells to produce an increased amount of Dpb3 polypeptides compared to the parental cells, wherein the modified cells produce during fermentation an increased amount of alcohol compared to the amount of alcohol produced by the parental cells under identical fermentation conditions.

2. In some embodiments of the modified cells of paragraph 1, the genetic alteration comprises the introduction into the parental cells of a nucleic acid capable of directing the expression of a Dpb3 polypeptide to a level above that of the parental cell grown under equivalent conditions.

3. In some embodiments of the modified cells of paragraph 1, the genetic alteration comprises the introduction of an expression cassette for expressing a Dpb3 polypeptide.

4. In some embodiments of the modified cells of paragraph 1, the genetic alteration comprises the introduction of an exogenous YBR278w c gene.

5. In some embodiments of the modified cells of paragraph 2, the genetic alteration comprises the introduction of a stronger promoter in an endogenous YBR278w c gene.

6. In some embodiments of the modified cells of any of paragraphs 1-5, the amount of increase in the expression of the Dpb3 polypeptide is at least about 30-fold compared to the level expression in the parental cells grown under equivalent conditions.

7. In some embodiments of the modified cells of any of paragraphs 1-5, the amount of increase in the production of mRNA encoding the Dpb3 polypeptide is at least about 30-fold compared to the level in the parental cells grown under equivalent conditions.

8. In some embodiments, the modified cells of paragraphs 1-7, further comprise disruption of the YJL065c gene.

9. In some embodiments of the modified cells of any of paragraphs 1-8, the cells produce a reduced amount of functional Dls1 polypeptides.

10. In some embodiments of the modified cells of any of paragraphs 1-9, the cells do not produce Dls1 polypeptides.

11. In some embodiments of the modified cells of any of paragraphs 1-10, the cells further comprise an exogenous gene encoding a carbohydrate processing enzyme.

12. In some embodiments, the modified cells of any of paragraphs 1-11, further comprise an alteration in the glycerol pathway and/or the acetyl-CoA pathway.

13. In some embodiments, the modified cells of any of paragraphs 1-12, further comprise an alternative pathway for making ethanol.

14. In some embodiments of the modified cells of paragraphs 1-13, the cells are of a Saccharomyces spp.

15. In another aspect, a method for increasing the production of alcohol from yeast cells grown on a carbohydrate substrate is provided, comprising: introducing into parental yeast cells a genetic alteration that increases the production of Dpb3 polypeptides compared to the amount produced in the parental cells.

16. In some embodiments of the method of paragraph 15, the cells having the introduced genetic alteration are the modified cells are the cells of any of paragraphs 1-15.

17. In some embodiments of the method of paragraph 15, the cells having the introduced genetic alteration that increases the production of Dpb3 polypeptides further comprises a genetic alteration that reduces the amount of functional Dls1 polypeptides produced compared to the parental cells.

These and other aspects and embodiments of present compositions and methods will be apparent from the description, including any accompanying Drawings.

### DETAILED DESCRIPTION

### I. Overview

The present compositions and methods relate to genetically-modified yeast cells that overexpress Dpb3 polypeptides compared to otherwise-identical parental cells. The modified cells produce an increased amount of alcohol compared to the parental cells, which phenotypes are separately, and in combination, advantageous in the production of fuel alcohol. Aspect and embodiments of the composition and methods are described in detail, herein.

### II. Definitions

Prior to describing the modified cells and methods of use in detail, the following terms are defined for clarity. Terms not defined should be accorded their ordinary meanings as used in the relevant art.

As used herein, “alcohol” refer to an organic compound in which a hydroxyl functional group (—OH) is bound to a saturated carbon atom.

As used herein, “yeast cells” yeast strains, or simply “yeast” refer to organisms from the phyla Ascomycota and Basidiomycota. Exemplary yeast is budding yeast from the order Saccharomycetales. Particular examples of yeast are Saccharomyces spp., including but not limited to S. cerevisiae. Yeast include organisms used for the production of fuel alcohol as well as organisms used for the production of potable alcohol, including specialty and proprietary yeast strains used to make distinctive-tasting beers, wines, and other fermented beverages.

As used herein, the phrase “variant yeast cells,” “modified yeast cells,” or similar phrases (see above), refer to yeast that include genetic modifications and characteristics described herein. Variant/modified yeast do not include naturally occurring yeast.

As used herein, the phrase “substantially free of an activity,” or similar phrases, means that a specified activity is either undetectable in an admixture or present in an amount that would not interfere with the intended purpose of the admixture.

As used herein, the terms “polypeptide” and “protein” (and their respective plural forms) are used interchangeably to refer to polymers of any length comprising amino acid residues linked by peptide bonds. The conventional one-letter or three-letter codes for amino acid residues are used herein and all sequence are presented from an N-terminal to C-terminal direction. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, functionally and/or structurally similar proteins are considered to be “related proteins.” Such proteins can be derived from organisms of different genera and/or species, or even different classes of organisms (e.g., bacteria and fungi). Related proteins also encompass homologs determined by primary sequence analysis, determined by secondary or tertiary structure analysis, or determined by immunological cross-reactivity.

As used herein, the term “homologous protein” or “homolog” refers to a protein that has similar activity and/or structure to a reference protein. It is not intended that homologs necessarily be evolutionarily related. Thus, it is intended that the term encompass the same, similar, or corresponding enzyme(s) (i.e., in terms of structure and function) obtained from different organisms. In some embodiments, it is desirable to identify a homolog that has a quaternary, tertiary and/or primary structure similar to the reference protein. In some embodiments, homologous proteins induce similar immunological response(s) as a reference protein. In some embodiments, homologous proteins are engineered to produce enzymes with desired activity(ies).

The degree of homology between sequences can be determined using any suitable method known in the art (see, e.g., Smith and Waterman (1981) Adv. Appl. Math. 2:482; Needleman and Wunsch (1970) J. Mol. Biol., 48:443; Pearson and Lipman (1988) Proc. Natl. Acad. Sci. USA 85:2444; programs such as GAP, BESTFIT, FASTA, and TFASTA in the Wisconsin Genetics Software Package (Genetics Computer Group, Madison, Wis.); and Devereux et al. (1984) Nucleic Acids Res. 12:387-95).

For example, PILEUP is a useful program to determine sequence homology levels. PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Doolittle, (Feng and Doolittle (1987) J. Mol. Evol. 35:351-60). The method is similar to that described by Higgins and Sharp ((1989) CABIOS 5:151-53). Useful PILEUP parameters including a default gap weight of 3.00, a default gap length weight of 0.10, and weighted end gaps. Another example of a useful algorithm is the BLAST algorithm, described by Altschul et al. ((1990) J. Mol. Biol. 215:403-10) and Karlin et al. ((1993) Proc. Natl. Acad. Sci. USA 90:5873-87). One particularly useful BLAST program is the WU-BLAST-2 program (see, e.g., Altschul et al. (1996) Meth. Enzymol. 266:460-80). Parameters “W,” “T,” and “X” determine the sensitivity and speed of the alignment. The BLAST program uses as defaults a word-length (W) of 11, the BLOSUM62 scoring matrix (see, e.g., Henikoff and Henikoff (1989) Proc. Natl. Acad. Sci. USA 89:10915) alignments (B) of 50, expectation (E) of 10, M′5, N′-4, and a comparison of both strands.

As used herein, the phrases “substantially similar” and “substantially identical,” in the context of at least two nucleic acids or polypeptides, typically means that a polynucleotide or polypeptide comprises a sequence that has at least about 70% identity, at least about 75% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or even at least about 99% identity, or more, compared to the reference (i.e., wild-type) sequence. Percent sequence identity is calculated using CLUSTAL W algorithm with default parameters. See Thompson et al. (1994) Nucleic Acids Res. 22:4673-4680. Default parameters for the CLUSTAL W algorithm are:

Gap opening penalty: 10.0

Gap extension penalty: 0.05

Protein weight matrix: BLOSUM series

DNA weight matrix: IUB

Delay divergent sequences %: 40

Gap separation distance: 8

DNA transitions weight: 0.50

List hydrophilic residues: GPSNDQEKR

Use negative matrix: OFF

Toggle Residue specific penalties: ON

Toggle hydrophilic penalties: ON

Toggle end gap separation penalty OFF.

Another indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution. Another indication that two nucleic acid sequences are substantially identical is that the two molecules hybridize to each other under stringent conditions (e.g., within a range of medium to high stringency).

As used herein, the term “gene” is synonymous with the term “allele” in referring to a nucleic acid that encodes and directs the expression of a protein or RNA. Vegetative forms of filamentous fungi are generally haploid, therefore a single copy of a specified gene (i.e., a single allele) is sufficient to confer a specified phenotype.

As used herein, “expressing a polypeptide” and similar terms, refer to the cellular process of producing a polypeptide using the translation machinery (e.g., ribosomes) of the cell.

As used herein, “overexpressing a polypeptide,” “increasing the expression of a polypeptide,” and similar terms, refer to expressing a polypeptide at higher-than-normal levels compared to those observed with parental or “wild-type cells that do not include a specified genetic modification.

As used herein, an “expression cassette” refers to a nucleic acid that includes an amino acid coding sequence, promoters, terminators, and other nucleic acid sequence needed to allow the encoded polypeptide to be produced in a cell. Expression cassettes can be exogenous (i.e., introduced into a cell) or endogenous (i.e., extant in a cell).

As used herein, the terms “wild-type” and “native” are used interchangeably and refer to genes proteins or strains found in nature.

As used herein, the term “protein of interest” refers to a polypeptide that is desired to be expressed in modified yeast. Such a protein can be an enzyme, a substrate-binding protein, a surface-active protein, a structural protein, a selectable marker, or the like, and can be expressed at high levels. The protein of interest is encoded by a modified endogenous gene or a heterologous gene (i.e., gene of interest”) relative to the parental strain. The protein of interest can be expressed intracellularly or as a secreted protein.

As used herein, “deletion of a gene,” refers to its removal from the genome of a host cell. Where a gene includes control elements (e.g., enhancer elements) that are not located immediately adjacent to the coding sequence of a gene, deletion of a gene refers to the deletion of the coding sequence, and optionally adjacent enhancer elements, including but not limited to, for example, promoter and/or terminator sequences, but does not require the deletion of non-adjacent control elements.

As used herein, “disruption of a gene” refers broadly to any genetic or chemical manipulation, i.e., mutation, that substantially prevents a cell from producing a function gene product, e.g., a protein, in a host cell. Exemplary methods of disruption include complete or partial deletion of any portion of a gene, including a polypeptide-coding sequence, a promoter, an enhancer, or another regulatory element, or mutagenesis of the same, where mutagenesis encompasses substitutions, insertions, deletions, inversions, and combinations and variations, thereof, any of which mutations substantially prevent the production of a function gene product. A gene can also be disrupted using RNAi, antisense, or any other method that abolishes gene expression. A gene can be disrupted by deletion or genetic manipulation of non-adjacent control elements.

As used herein, the terms “genetic manipulation” and “genetic alteration” are used interchangeably and refer to the alteration/change of a nucleic acid sequence. The alteration can include but is not limited to a substitution, deletion, insertion or chemical modification of at least one nucleic acid in the nucleic acid sequence.

As used herein, a “functional polypeptide/protein” is a protein that possesses an activity, such as an enzymatic activity, a binding activity, a surface-active property, or the like, and which has not been mutagenized, truncated, or otherwise-modified to abolish or reduce that activity. Functional polypeptides can be thermostable or thermolabile, as specified.

As used herein, “a functional gene” is a gene capable of being used by cellular components to produce an active gene product, typically a protein. Functional genes are the antithesis of disrupted genes, which are modified such that they cannot be used by cellular components to produce an active gene product, or have a reduced ability to be used by cellular components to produce an active gene product.

As used herein, yeast cells have been “modified to prevent the production of a specified protein” if they have been genetically or chemically altered to prevent the production of a functional protein/polypeptide that exhibits an activity characteristic of the wild-type protein. Such modifications include, but are not limited to, deletion or disruption of the gene encoding the protein (as described, herein), modification of the gene such that the encoded polypeptide lacks the aforementioned activity, modification of the gene to affect post-translational processing or stability, and combinations, thereof.

As used herein, the term “paralog” refers to homologous genes that are the result of a duplication event.

As used herein, “attenuation of a pathway” or “attenuation of the flux through a pathway” i.e., a biochemical pathway, refers broadly to any genetic or chemical manipulation that reduces or completely stops the flux of biochemical substrates or intermediates through a metabolic pathway. Attenuation of a pathway may be achieved by a variety of well-known methods. Such methods include but are not limited to: complete or partial deletion of one or more genes, replacing wild-type alleles of these genes with mutant forms encoding enzymes with reduced catalytic activity or increased Km values, modifying the promoters or other regulatory elements that control the expression of one or more genes, engineering the enzymes or the mRNA encoding these enzymes for a decreased stability, misdirecting enzymes to cellular compartments where they are less likely to interact with substrate and intermediates, the use of interfering RNA, and the like.

As used herein, “aerobic fermentation” refers to growth in the presence of oxygen.

As used herein, “anaerobic fermentation” refers to growth in the absence of oxygen.

As used herein, the singular articles “a,” “an,” and “the” encompass the plural referents unless the context clearly dictates otherwise. All references cited herein are hereby incorporated by reference in their entirety. The following abbreviations/acronyms have the following meanings unless otherwise specified:
° C. degrees Centigrade
AA α-amylase
bp base pairs
DNA deoxyribonucleic acid
DP degree of polymerization
ds or DS dry solids
EtOH ethanol
g or gm gram
g/L grams per liter
GA glucoamylase
GAU/g ds glucoamylase units per gram dry solids
H₂O water
HPLC high performance liquid chromatography
hr or h hour
kg kilogram
M molar
mg milligram
mL or ml milliliter
ml/min milliliter per minute
mM millimolar
N normal
nm nanometer
PCR polymerase chain reaction
ppm parts per million
RNA ribonucleic acid
Δ relating to a deletion
μg microgram
μL and μl microliter
μM micromolar

### III. Modified Yeast Cells Having Increased Dpb3 Expression

In one aspect, modified yeast cells are provided, the modified cells having a genetic alteration that results in the production of increased amounts of Dpb3 polypeptides compared to corresponding (i.e., otherwise-identical) parental cells. Dpb3 is an approximately 200-amino acid DNA polymerase II epsilon subunit originally identified in Saccharomyces cerevisiae (see, e.g., Araki, H. et al. (1991) Nucleic Acids Res. 19:4867-72).

Applicants have discovered that yeast cells overexpressing Dpb3 polypeptides produce an increased amount of alcohol compared to otherwise-identical parental cells. Increased alcohol production is desirable as it improves the output of alcohol production facilities and represents better carbon utilization from starting carbohydrate-containing materials.

In some embodiments, the increase in the amount of Dpb3 polypeptides produced by the modified cells is an increase of at least 100%, at least 200%, at least 300%, at least 400%, at least 500%, at least 600%, at least 700%, at least 800%, at least 900%, at least 1,000%, or even at least 3,000%, or more, compared to the amount of Dpb3 polypeptides produced by parental cells grown under the same conditions.

In some embodiments, the increase in the strength of the promoter used to control expression of the Dpb3 polypeptide produced by the modified cells is at least 30-fold, at least 40-fold, at least 50-fold, at least 60-fold, at least 70-fold, at least 75-fold, at least 80-fold, at least 90-fold, at least 100-fold, at least 110-fold, at least 120-fold, at least 130-fold, or even at least 135%-fold, or more, compared to strength of the native promoter controlling Dpb3 expression, based on the amount of mRNA produced.

In some embodiments, the increase in the amount of Dpb3 polypeptides produced by the modified cells is an increase of at least 30-fold, at least 40-fold, at least 50-fold, at least 60-fold, at least 70-fold, at least 75-fold, at least 80-fold, at least 90-fold, at least 100-fold, at least 110-fold, at least 120-fold, at least 130-fold, or even at least 135%-fold, or more, compared to the amount of Dpb3 polypeptides produced by parental cells grown under the same conditions.

In some embodiments, the increase in alcohol production by the modified cells is an increase of at least 1%, at least 2%, at least 3%, at least 4%, at least 5%, at least 6%, at least 7%, or more, compared to the amount of alcohol produced by parental cells grown under the same conditions.

Preferably, increased Dpb3 expression is achieved by genetic manipulation using sequence-specific molecular biology techniques, as opposed to chemical mutagenesis, which is generally not targeted to specific nucleic acid sequences. However, chemical mutagenesis is not excluded as a method for making modified yeast cells.

In some embodiments, the present compositions and methods involve introducing into yeast cells a nucleic acid capable of directing the overexpression, or increased expression, of a Dpb3 polypeptide. Particular methods include but are not limited to (i) introducing an exogenous expression cassette for producing the polypeptide into a host cell, optionally in addition to an endogenous expression cassette, (ii) substituting an exogenous expression cassette with an endogenous cassette that allows the production of an increased amount of the polypeptide, (iii) modifying the promoter of an endogenous expression cassette to increase expression, and/or (iv) modifying any aspect of the host cell to increase the half-life of the polypeptide in the host cell.

In some embodiments, the parental cell that is modified already includes a gene of interest, such as a gene encoding a selectable marker, carbohydrate-processing enzyme, or other polypeptide. In some embodiments, a gene of introduced is subsequently introduced into the modified cells.

The amino acid sequence of the exemplified S. cerevisiae Dpb3 polypeptide (i.e., EMBLE Accession No. Z36146.1) is shown, below, as SEQ ID NO: 1:

MSNLVKEKAP VFPISKVKKI AKCDPEYVIT SNVAISATAF
AAELPVQNLV EESLVLAQLN SKGKTSLRLS LNSIEECVEK
RDNFRFLEDA IKQLKKNSAL DKKRELNMQP GRSDQEVVIE
EPELHEDDGV EEEEEEDEVS EEEEPVHNEE LLDDSKDQQN
DKSTRSVASL LSRFQYKSAL DVGEHSDSSD IEVDHIKSTD P

The NCBI database includes over 100 entries for S. cerevisiae Dpb3 polypeptides and natural variations in the amino acid sequence are not expected to affect its function.

Based on such BLAST and Clustal W data, it is apparent that the exemplified S. cerevisiae Dpb3 polypeptide shares a high degree of sequence identity to polypeptides from other organisms, and overexpression of functionally and/or structurally similar proteins, homologous proteins and/or substantially similar or identical proteins, is expected to produce similar beneficial results.

In particular embodiments of the present compositions and methods, the amino acid sequence of the Dpb3 polypeptide that is overexpressed in modified yeast cells has at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or even at least about 99% identity, to SEQ ID NO: 1.

### IV. Modified Yeast Cells Having Increased Dpb3 Expression and Reduced Dls1 Expression

Dls1, encoded by YJL065c, is a 167-amino acid polypeptide subunit of the ISW2 yeast chromatin accessibility complex (yCHRAC), which contains Isw2, Itc1, Dpb3-like subunit (Dls1), and Dpb4 (see, e.g., Peterson, C. L. (1996) Curr. Opin. Genet. Dev. 6:171-75 and Winston, F. and Carlson, M. (1992) Trends Genet. 8:387-91). Applicants have determined that yeast having a genetic alteration that reduces the amount of functional Dls1 in the cell, in the absence of other genetic modifications, exhibit increased robustness in an alcohol fermentation, allowing higher-temperature, and potentially shorter, fermentations (data not shown).

Dpb3 was initially identified as a paralog of Dls1. However, as described herein, whereas genetic alterations that reduced the amount of functional Dls1 in the cell increased the robustness of alcohol production, the opposite is true of genetic alterations that reduced the amount of functional Dpb3 polypeptide. Accordingly, in some embodiments of the present compositions and methods, modifications in yeast that increase Dpb3 expression are combined with modifications that reduce or eliminate the amount of functional of Dls1 in the cell, further increasing the amount of alcohol produced by the resulting engineered yeast.

Reduction in the amount of functional Dls1 produced in a cell can be accomplished by disruption of the YJL065c gene. Disruption of the YJL065c gene can be performed using any suitable methods that substantially prevent expression of a function YJL065c gene product, i.e., Dls1. Exemplary methods of disruption as are known to one of skill in the art include but are not limited to: complete or partial deletion of the YJL065c gene, including complete or partial deletion of, e.g., the Dls1-coding sequence, the promoter, the terminator, an enhancer, or another regulatory element; and complete or partial deletion of a portion of the chromosome that includes any portion of the YJL065c gene. Particular methods of disrupting the YJL065c gene include making nucleotide substitutions or insertions in any portion of the YJL065c gene, e.g., the Dls1-coding sequence, the promoter, the terminator, an enhancer, or another regulatory element. Preferably, deletions, insertions, and/or substitutions (collectively referred to as mutations) are made by genetic manipulation using sequence-specific molecular biology techniques, as opposed to by chemical mutagenesis, which is generally not targeted to specific nucleic acid sequences. Nonetheless, chemical mutagenesis can, in theory, be used to disrupt the YJL065c gene.

Mutations in the YJL065c gene can reduce the efficiency of the YJL065c promoter, reduce the efficiency of a YJL065c enhancer, interfere with the splicing or editing of the YJL065c mRNA, interfere with the translation of the YJL065c mRNA, introduce a stop codon into the YJL065c-coding sequence to prevent the translation of full-length tYJL065c protein, change the coding sequence of the Dls1 protein to produce a less active or inactive protein or reduce Dls1 interaction with other nuclear protein components, or DNA, change the coding sequence of the Dls1 protein to produce a less stable protein or target the protein for destruction, cause the Dls1 protein to misfold or be incorrectly modified (e.g., by glycosylation), or interfere with cellular trafficking of the Dls1 protein. In some embodiments, these and other genetic manipulations act to reduce or prevent the expression of a functional Dls1 protein, or reduce or prevent the normal biological activity of Dls1.

In some embodiments, the present modified cells include genetic manipulations that reduce or prevent the expression of a functional Dls1 protein, or reduce or prevent the normal biological activity of Dls1, as well as additional mutations that reduce or prevent the expression of a functional Isw2, Itc1, or Dpb4 proteins or reduce or prevent the normal biological activity of Isw2, Itc1, or Dpb4 proteins. In some embodiments, the present modified cells include genetic manipulations that reduce or prevent the expression of a functional Dls1 protein, or reduce or prevent the normal biological activity of Dls1, while having no additional mutations that reduce or prevent the expression of a functional Isw2, Itc1, or Dpb4 proteins or reduce or prevent the normal biological activity of Isw2, Itc1, or Dpb4 proteins.

The amino acid sequence of the exemplified S. cerevisiae Dls1 polypeptide is shown, below, as SEQ ID NO: 2:

MNNETSGKET ASAPLCSPKL PVEKVQRIAK NDPEYMDTSD
DAFVATAFAT EFFVQVLTHE SLHRQQQQQQ QQVPPLPDEL
TLSYDDISAA IVHSSDGHLQ FLNDVIPTTK NLRLLVEENR
VRYTTSVMPP NEVYSAYVVN DTAPKPNIVE IDLDNDEDD
EDVTDQE

Based on such BLAST and Clustal W data, it is apparent that the exemplified S. cerevisiae Dls1 polypeptide (SEQ ID NO: 2) share a very high degree of sequence identity to other known S. cerevisiae Dls1 polypeptides, as well as Dls1 polypeptides from other Saccharomyces spp. The present compositions and methods, are therefore, fully expected to be applicable to yeast cells containing such structurally similar polypeptides, as well as other related proteins, homologs, and functionally similar polypeptides.

In some embodiments of the present compositions and methods, the amino acid sequence of the Dls1 protein that is disrupted has an overall amino acid sequence identity to the amino acid sequence of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or even at least about 99% identity, to SEQ ID NO: 2

Preferably, disruption of the YJL065c gene is performed by genetic manipulation using sequence-specific molecular biology techniques, as opposed to chemical mutagenesis, which is generally not targeted to specific nucleic acid sequences. However, chemical mutagenesis is not excluded as a method for making modified yeast cells.

In some embodiments, the decrease in the amount of functional Dls1 polypeptide in the modified cells is a decrease of at least 30%, at least 40%, at least 50%, at least 60%, at least 70%, at least 80%, at least 90%, at least 95%, at least 98%, at least 99%, or more, compared to the amount of functional Dls1 polypeptide in parental cells growing under the same conditions. In some embodiments, the reduction of expression of functional Dls1 protein in the modified cells is a reduction of at least 30%, at least 40%, at least 50%, at least 60%, at least 70%, at least 80%, at least 90%, at least 95%, at least 98%, at least 99%, or more, compared to the amount of functional Dls1 polypeptide in parental cells growing under the same conditions.

In some embodiments, the additional increase in alcohol in the modified cells, compared to cells that only overexpress Dpb3, is an increase of at least 0.2%, at least 0.4%, at least 0.6%, at least 0.8%, at least 1.0%, or more.

### V. Combination of Increased Dpb3 Expression with Other Mutations that Affect Alcohol Production

In some embodiments, in addition to overexpressing Dpb3 polypeptides, optionally in combination with reducing the expression of functional Dls1 polypeptides, the present modified yeast cells further include additional modifications that affect alcohol production.

In particular embodiments the modified yeast cells include an artificial or alternative pathway resulting from the introduction of a heterologous phosphoketolase gene and a heterologous phosphotransacetylase gene. An exemplary phosphoketolase can be obtained from Gardnerella vaginalis (UniProt/TrEMBL Accession No.: WP_016786789). An exemplary phosphotransacetylase can be obtained from Lactobacillus plantarum (UniProt/TrEMBL Accession No.: WP_003641060).

The modified cells may further include mutations that result in attenuation of the native glycerol biosynthesis pathway, which are known to increase alcohol production. Methods for attenuation of the glycerol biosynthesis pathway in yeast are known and include reduction or elimination of endogenous NAD-dependent glycerol 3-phosphate dehydrogenase (GPD) or glycerol phosphate phosphatase activity (GPP), for example by disruption of one or more of the genes GPD1, GPD2, GPP1 and/or GPP2. See, e.g., U.S. Pat. No. 9,175,270 (Elke et al.), U.S. Pat. No. 8,795,998 (Pronk et al.) and U.S. Pat. No. 8,956,851 (Argyros et al.).

The modified yeast may further feature increased acetyl-CoA synthase (also referred to acetyl-CoA ligase) activity (EC 6.2.1.1) to scavenge (i.e., capture) acetate produced by chemical or enzymatic hydrolysis of acetyl-phosphate (or present in the culture medium of the yeast for any other reason) and converts it to Ac-CoA. This avoids the undesirable effect of acetate on the growth of yeast cells and may further contribute to an improvement in alcohol yield. Increasing acetyl-CoA synthase activity may be accomplished by introducing a heterologous acetyl-CoA synthase gene into cells, increasing the expression of an endogenous acetyl-CoA synthase gene and the like. A particularly useful acetyl-CoA synthase for introduction into cells can be obtained from Methanosaeta concilii (UniProt/TrEMBL Accession No.: WP_013718460). Homologs of this enzymes, including enzymes having at least 85%, at least 90%, at least 92%, at least 95%, at least 97%, at least 98% and even at least 99% amino acid sequence identity to the aforementioned acetyl-CoA synthase from Methanosaeta concilii, are also useful in the present compositions and methods.

In some embodiments the modified cells may further include a heterologous gene encoding a protein with NAD⁺-dependent acetylating acetaldehyde dehydrogenase activity and/or a heterologous gene encoding a pyruvate-formate lyase. The introduction of such genes in combination with attenuation of the glycerol pathway is described, e.g., in U.S. Pat. No. 8,795,998 (Pronk et al.). However, in most embodiments of the present compositions and methods, the introduction of an acetylating acetaldehyde dehydrogenase and/or a pyruvate-formate lyase is not required because the need for these activities is obviated by the attenuation of the native biosynthetic pathway for making Ac-CoA that contributes to redox cofactor imbalance. Accordingly, embodiments of the present compositions and methods expressly lack a heterologous gene(s) encoding an acetylating acetaldehyde dehydrogenase, a pyruvate-formate lyase or both.

In some embodiments, the present modified yeast cells further comprise a butanol biosynthetic pathway. In some embodiments, the butanol biosynthetic pathway is an isobutanol biosynthetic pathway. In some embodiments, the isobutanol biosynthetic pathway comprises a polynucleotide encoding a polypeptide that catalyzes a substrate to product conversion selected from the group consisting of: (a) pyruvate to acetolactate; (b) acetolactate to 2,3-dihydroxyisovalerate; (c) 2,3-dihydroxyisovalerate to 2-ketoisovalerate; (d) 2-ketoisovalerate to isobutyraldehyde; and (e) isobutyraldehyde to isobutanol. In some embodiments, the isobutanol biosynthetic pathway comprises polynucleotides encoding polypeptides having acetolactate synthase, keto acid reductoisomerase, dihydroxy acid dehydratase, ketoisovalerate decarboxylase, and alcohol dehydrogenase activity.

In some embodiments, the present modified yeast cells may further overexpress a sugar transporter-like (STL1) polypeptide to increase the uptake of glycerol (see, e.g., Ferreira et al. (2005) Mol Biol Cell 16:2068-76; Dušková et al. (2015) Mol Microbiol 97:541-59 and WO 2015023989 A1).

In some embodiments, the modified yeast cells comprising a butanol biosynthetic pathway further comprise a modification in a polynucleotide encoding a polypeptide having pyruvate decarboxylase activity. In some embodiments, the yeast cells comprise a deletion, mutation, and/or substitution in an endogenous polynucleotide encoding a polypeptide having pyruvate decarboxylase activity. In some embodiments, the polypeptide having pyruvate decarboxylase activity is selected from the group consisting of: PDC1, PDC5, PDC6, and combinations thereof. In some embodiments, the yeast cells further comprise a deletion, mutation, and/or substitution in one or more endogenous polynucleotides encoding FRA2, ALD6, ADH1, GPD2, BDH1, and YMR226C.

### Goi Section

### VI. Combination of Increased Dpb3 Expression with Other Beneficial Mutations

In some embodiments, in addition to overexpressing Dpb3 polypeptides, optionally in combination with other genetic modifications that benefit alcohol production, the present modified yeast cells further include any number of additional genes of interest encoding proteins of interest. Additional genes of interest may be introduced before, during, or after genetic manipulations that result in overexpression of Dpb3 polypeptides. Proteins of interest, include selectable markers, carbohydrate-processing enzymes, and other commercially-relevant polypeptides, including but not limited to an enzyme selected from the group consisting of a dehydrogenase, a transketolase, a phosphoketolase, a transladolase, an epimerase, a phytase, a xylanase, a β-glucanase, a phosphatase, a protease, an α-amylase, a β-amylase, a glucoamylase, a pullulanase, an isoamylase, a cellulase, a trehalase, a lipase, a pectinase, a polyesterase, a cutinase, an oxidase, a transferase, a reductase, a hemicellulase, a mannanase, an esterase, an isomerase, a pectinases, a lactase, a peroxidase and a laccase. Proteins of interest may be secreted, glycosylated, and otherwise-modified.

### VII. Use of the Modified Yeast for Increased Alcohol Production

The present compositions and methods include methods for increasing alcohol production and/or reducing glycerol production, in fermentation reactions. Such methods are not limited to a particular fermentation process. The present engineered yeast is expected to be a “drop-in” replacement for convention yeast in any alcohol fermentation facility. While primarily intended for fuel alcohol production, the present yeast can also be used for the production of potable alcohol, including wine and beer.

### VIII. Yeast Cells Suitable for Modification

Yeasts are unicellular eukaryotic microorganisms classified as members of the fungus kingdom and include organisms from the phyla Ascomycota and Basidiomycota. Yeast that can be used for alcohol production include, but are not limited to, Saccharomyces spp., including S. cerevisiae, as well as Kluyveromyces, Lachancea and Schizosaccharomyces spp. Numerous yeast strains are commercially available, many of which have been selected or genetically engineered for desired characteristics, such as high alcohol production, rapid growth rate, and the like. Some yeasts have been genetically engineered to produce heterologous enzymes, such as glucoamylase or α-amylase.

### IX. Substrates and Products

Alcohol production from a number of carbohydrate substrates, including but not limited to corn starch, sugar cane, cassava, and molasses, is well known, as are innumerable variations and improvements to enzymatic and chemical conditions and mechanical processes. The present compositions and methods are believed to be fully compatible with such substrates and conditions.

Alcohol fermentation products include organic compound having a hydroxyl functional group (—OH) is bound to a carbon atom. Exemplary alcohols include but are not limited to methanol, ethanol, n-propanol, isopropanol, n-butanol, isobutanol, n-pentanol, 2-pentanol, isopentanol, and higher alcohols. The most commonly made fuel alcohols are ethanol, and butanol.

These and other aspects and embodiments of the present yeast strains and methods will be apparent to the skilled person in view of the present description. The following examples are intended to further illustrate, but not limit, the compositions and methods.

### EXAMPLES

### Example 1. Deletion of the YBR278w Gene in S. cerevisiae

Dpb3, encoded by YBR278w, is a paralog of Dls1, encoded by YJL065c (Iida, T. and Araki, H. (2004) Mol Cell Biol, 24:217-27). As described, above reducing the amount of Dls1 produced in yeast cells, with no other genetic modifications to the cells, increased alcohol production (data not shown). An initial experiment was performed to determine if reducing the amount of Dpb3 produced in yeast cells also increased alcohol production.

Using standard yeast molecular biology techniques, the YBR278w gene was disrupted by deleting essentially the entire coding sequence for Dpb3. All procedures were based on the publically available nucleic acid sequence of YBR278w, which is provided below as SEQ ID NO 3 (5′ to 3′):

ATGTCCAACTTAGTTAAAGAAAAAGCACCTGTCTTTCCTATATCTAAAGT
AAAGAAGATTGCCAAATGCGACCCCGAATACGTAATTACATCTAATGTAG
CTATATCAGCGACCGCATTCGCTGCTGAGTTATTTGTACAGAATCTCGTC
GAAGAATCGCTGGTCTTAGCACAACTGAATTCGAAAGGAAAGACAAGCCT
ACGATTAAGCCTAAATTCTATAGAAGAATGTGTCGAAAAAAGAGATAATT
TCAGGTTTCTAGAGGATGCCATTAAACAACTGAAGAAGAATAGCGCACTC
GACAAGAAGAGAGAACTAAACATGCAACCGGGTCGGAGCGATCAAGAGGT
TGTTATAGAAGAGCCTGAATTGCATGAGGATGATGGTGTAGAGGAAGAAG
AAGAAGAAGACGAGGTATCCGAAGAAGAAGAGCCCGTACACAATGAAGAA
CTTCTTGATGATAGTAAAGATCAACAAAATGATAAATCCACGCGCAGTGT
GGCAAGCTTGCTGTCGAGATTCCAGTATAAATCCGCACTAGACGTAGGAG
AACACTCCGACTCTTCTGATATCGAAGTTGACCATACGAAAAGCACCGAT
CCTTAG

The host yeast used to make the modified yeast cells was commercially available FERMAX™ Gold (Martrex, Inc., Chaska, Minn., USA, herein “FG”). Deletion of the YBR278w gene was confirmed by colony PCR. The modified yeast was grown in non-selective media to remove the plasmid conferring Kanamycin resistance used to select transformants, resulting in modified yeast that required no growth supplements compared to the parental yeast. Three independent modified strains, designated DPB3del-1, DPB3del-2 and DPB3del-3, were selected for further study.

### Example 2: Ethanol Production by Modified Yeast with Reduced Expression of Dpb3

DPB3del-1, DPB3del-2 and DPB3del-3 yeast harboring the deletion of the YBR278w gene were tested for their ability to produce ethanol compared to benchmark yeast (i.e., FERMAX™ Gold), which is wild-type for the YBR278w gene) in liquefact at 34 and 37° C. Liquefact (i.e., corn flour slurry having a dry solid (ds) value of 35% was prepared by adding 600 ppm urea, 0.124 SAPU/g ds FERMGEN™ 2.5× (an acid fungal protease), 0.33 GAU/g ds CS4 (a variant of Trichoderma reesei glucoamylase) and 1.46 SSCU/g ds Aspergillus kawachii α-amylase at pH 4.8.

2.5 grams of liquefact was weighted into 10 ml vessels and inoculated with fresh overnight cultures from colonies of the modified strains or FG strain and incubated at different temperatures. Samples were harvested by centrifugation at 65 hrs, filtered through 0.2 μm filters, and analyzed for glucose and ethanol content by HPLC (Agilent Technologies 1200 series) using Bio-Rad Aminex HPX-87H columns at 55° C., with an isocratic flow rate of 0.6 ml/min in 0.01 N H₂SO₄ eluent. The results of the analyses are shown in Table 1. Ethanol production is reported with reference to the FG strain.

TABLE 1
Analysis of fermentation broth following fermentation

| Temperature | | Glucose | Ethanol | Ethanol |
|---|---|---|---|---|
| (° C.) | Strain | (g/L) | (g/L) | compared to FG |
| 34 | FG | 17.3 | 139.31 | 1 |
| 34 | DPB3del-1 | 25.63 | 135.71 | 0.974 |
| 34 | DPB3del-2 | 26.59 | 135.17 | 0.97 |
| 34 | DPB3del-3 | 25.55 | 135.32 | 0.971 |
| 37 | FG | 55.99 | 122.08 | 1 |
| 37 | DPB3del-1 | 78.58 | 108.4 | 0.888 |
| 37 | DPB3del-2 | 78.38 | 108.64 | 0.89 |
| 37 | DPB3del-3 | 78.81 | 108.17 | 0.886 |

Yeast harboring the deletion of the gene YBR278w c produced less ethanol compared to the reference strain, particularly at the elevated temperature.

### Example 3: Overexpression of Dpb3 in S. cerevisiae

The overexpression of Dpb3-1 in rice was to be found associated with heat tolerance (Hikaro Sato, H. et al. (2016) Plant Biotech J, 14:1756-67). An initial experiment was performed to determine if increasing the amount of Dpb3 in yeast also increase tolerance and alcohol production.

Four promoters with different strength were used: TMP1 (12-fold increase in expression), HTA1 (28-fold increase in expression), EFB1 (75-fold increase in expression) and FBA1 (135-fold increase in expression). Fold-increase in expression was based on the amount of mRNA produced (data not shown). Using standard techniques, the native promoter of DPB3 was swapped with each of the four promoters mentioned above in the aforementioned FG host yeast. The promoter swaps of the YBR278w gene was confirmed by colony PCR.

The modified yeast was grown in non-selective media to remove the plasmid conferring Kanamycin resistance used to select transformants, resulting in modified yeast that required no growth supplements compared to the parental yeast. Three independent strains were selected for each promoter-swap variant, named TMP1-DPB3, HTA1-DPB3, EFB1-DPB3 and FBA1-DPB3, which were used for further study.

### Example 4: Ethanol Production by Modified Yeast with Increased Expression of Dpb3

The yeast strains harboring the overexpression of the YBR278w gene were tested for their ability to produce ethanol compared to benchmark yeast (i.e., FERMAX™ Gold), which is wild-type for the YBR278w gene, in liquefact at 34 and 37° C. Liquefact (i.e., corn flour slurry having a dry solid (ds) value of 35% was prepared by adding 600 ppm urea, 0.124 SAPU/g ds FERMGEN™ 2.5× (an acid fungal protease), 0.33 GAU/g ds CS4 (a variant of Trichoderma reesei glucoamylase) and 1.46 SSCU/g ds AKAA (Aspergillus kawachii α-amylase) at pH 4.8.

2.5 grams of liquefact was weighted into 10 ml vessels and inoculated with fresh overnight cultures from colonies of the modified strains or FG strain and incubated at 34 C. Samples were harvested by centrifugation after 65 hrs, filtered through 0.2 μm filters, and analyzed for glucose and ethanol content by HPLC (Agilent Technologies 1200 series) using Bio-Rad Aminex HPX-87H columns at 55° C., with an isocratic flow rate of 0.6 ml/min in 0.01 N H₂SO₄ eluent. The results of the analyses are shown in Tables 2 to 5. Ethanol production is reported with reference to the FG strain.

TABLE 2
Analysis of fermentation broth following fermentation with yeast
harboring the TMP1, HTA1 and EFB1 promoters at 34° C.

| | Glucose | Ethanol | Ethanol |
|---|---|---|---|
| Strain | (g/L) | (g/L) | compared to FG |
| FG | 43.47 | 128.05 | 1 |
| TMP1-DPB3-1 | 42.85 | 127.49 | 0.996 |
| TMP1-DPB3-2 | 43.05 | 128.07 | 1 |
| TMP1-DPB3-3 | 43.13 | 128.33 | 1.002 |
| HTA1-DPB3-1 | 41.27 | 127.76 | 0.998 |
| HTA1-DPB3-2 | 41.92 | 127.98 | 0.999 |
| HTA1-DPB3-3 | 41.56 | 128.3 | 1.002 |
| EFB1-DPB3-1 | 24.81 | 136.35 | 1.065 |
| EFB1-DPB3-2 | 24.36 | 136.86 | 1.069 |
| EFB1-DPB3-3 | 25.78 | 135.12 | 1.063 |

TABLE 3
Analysis of fermentation broth following fermentation with
yeast harboring the FBA1 promoter at 34° C.

| | Glucose | Ethanol | Ethanol |
|---|---|---|---|
| Strain | (g/L) | (g/L) | compared to FG |
| FG | 28.25 | 132.8 | 1 |
| FBA1-DPB3-1 | 22.9 | 134.47 | 1.013 |
| FBA1-DPB3-2 | 22.06 | 135.24 | 1.018 |
| FBA1-DPB3-3 | 22.5 | 134.58 | 1.013 |

TABLE 4
Analysis of fermentation broth following fermentation with yeast
harboring the TMP1, HTA1 and EFB1 promoters at 37° C.

| | Glucose | Ethanol | Ethanol |
|---|---|---|---|
| Strain | (g/L) | (g/L) | compared to FG |
| FG | 69.43 | 118.73 | 1 |
| TMP1-DPB3-1 | 70.02 | 119.34 | 1.005 |
| TMP1-DPB3-2 | 71.27 | 119 | 1.002 |
| TMP1-DPB3-3 | 70.03 | 118.99 | 1.002 |
| HTA1-DPB3-1 | 69.71 | 118.98 | 1.002 |
| HTA1-DPB3-2 | 70.3 | 119.34 | 1.005 |
| HTA1-DPB3-3 | 69.62 | 119.39 | 1.006 |
| EFB1-DPB3-1 | 62.61 | 123.25 | 1.038 |
| EFB1-DPB3-2 | 59.89 | 124.12 | 1.045 |
| EFB1-DPB3-3 | 59.5 | 124.25 | 1.046 |

TABLE 5
Analysis of fermentation broth following fermentation with
yeast harboring the FBA1 promoter at 37° C.

| | Glucose | Ethanol | Ethanol |
|---|---|---|---|
| Strain | (g/L) | (g/L) | compared to FG |
| FG | 58.86 | 118.37 | 1 |
| FBA1-DPB3-1 | 56.18 | 119.9 | 1.013 |
| FBA1-DPB3-2 | 56.28 | 119.88 | 1.013 |
| FBA1-DPB3-3 | 55.82 | 119.87 | 1.013 |

Yeast harboring the YBR278w gene controlled by EFB1 and FBA1 promoters produced significantly (i.e., >1%) more ethanol compared to the reference strain, particularly at 34° C.

As shown in Table 6, yeast harboring the YBR278w gene controlled by the EFB1 promoter also produced significantly (i.e., ˜0.7%) more ethanol compared to the reference strain at a lower temperature of 33° C. and an a higher ds of 35.8.

TABLE 6
Analysis of fermentation broth following fermentation with yeast harboring the
EFB1 promoters at 32° C. and a higher ds

| | | | | | Ethanol | | Glucose |
|---|---|---|---|---|---|---|---|
| Temp. | | | Sampling | Ethanol | compared | Glucose | compared |
| (° C.) | DS (%) | Strain | time (hrs) | (g/L) | to FG | (g/L) | to FG |
| 32 | 35.8 | FG | 55 | 149.51 | 1 | 9.02 | 1 |
| 32 | 35.8 | EFB1-DPB3 | 55 | 150.4 | 1.007 | 5.91 | 0.65 |

Yeast harboring the different promoters was also tested for their ability to produce ethanol compared to the benchmark yeast in liquefact incubated according to the temperature ramp conditions shown in Table 7. Liquefact (i.e., corn flour slurry having a dry solid (ds) value of 35% was prepared by adding 600 ppm urea, 0.124 SAPU/g ds FERMGEN™ 2.5× (an acid fungal protease), 0.33 GAU/g ds CS4 (a variant of Trichoderma reesei glucoamylase) and 1.46 SSCU/g ds Aspergillus kawachii α-amylase at pH 4.8.

TABLE 7
Temperature ramp condition

| Time (hour) | Temperature (° C.) |
|---|---|
| 0-10 | 32 |
| 10-12 | 33 |
| 12-15 | 34 |
| 15-17 | 35 |
| 17-22 | 35.5 |
| 22-27 | 34.5 |
| 27-31 | 34 |
| 31-36 | 33.5 |
| 36-41 | 33 |
| 41-55 | 32.5 |
| 55-end | 32 |

30 grams of liquefact was weighted into 250 ml vessels and inoculated with fresh overnight cultures from colonies of the modified strain or FG strain under the temperature ramp conditions. A gas monitoring system (ANKOM Technology) was used to record the rate of fermentation based on cumulative pressure following CO₂ production over time. Samples were harvested by centrifugation, filtered through 0.2 μm filters, and analyzed for ethanol, glucose, acetate and glycerol content by HPLC (Agilent Technologies 1200 series) using Bio-Rad Aminex HPX-87H columns at 55° C., with an isocratic flow rate of 0.6 ml/min in 0.01 N H₂SO₄ eluent. A 2.5 μl sample injection volume was used. Calibration standards used for quantification included known amounts of DP4+, DP3, DP2, DP1, glycerol and ethanol. The results of the analyses are shown in Table 8. Ethanol increase is reported with reference to the FG strain.

TABLE 8
Analysis of fermentation broth following fermentation
under temperature ramp conditions

| | Glucose | Ethanol | Ethanol |
|---|---|---|---|
| Strain | (g/L) | (g/L) | compared to FG |
| FG | 24.57 | 137.21 | 1 |
| TMA1-DPB3 | 26.04 | 135.54 | 0.988 |
| HTA1-DPB3 | 24.27 | 136.93 | 0.988 |
| EFB1-DPB3 | 12.08 | 143.23 | 1.044 |
| FBA1-DPB3 | 22.85 | 137.83 | 1.005 |

Yeast harboring the YBR278w gene controlled by EFB1 promoter produced significantly (i.e., >4%) more ethanol compared to the reference strain under temperature ramp conditions. Based on the totality of the experimental data, it appears that at least a 30-fold increase in promoter strength, based on mRNA production, is required to realize a significant increase in alcohol production as a result of Dpb3 over expression. The corresponding increase in the amount of Dpb1 protein produced in the cells may be lower.

### Example 5: Overexpression of Dpb3 in Combination with Reduced Expression of Dls1

An experiment was performed to determine if increasing the amount of Dpb3 in combination with reducing the amount of Dls1 (encoded by the YJL065c gene) in yeast increase further increase tolerance and alcohol production compared to increase Dpb3, alone. Using standard techniques, the native promoter of Dpb3 was swapped with the EFB1 promoter in the aforementioned FG host yeast in which the deletion of YJL065c had been made. The promoter swap of the YBR278w gene was confirmed by colony PCR. Yeast harboring the deletion of YJL065c and the EFB1 promoter in front of Dpb3 genes were tested for their ability to produce ethanol compared to the benchmark yeast in liquefact incubated according to the temperature ramp conditions shown in Table 6. Liquefact (i.e., corn flour slurry having a dry solid (ds) value of 35% was prepared by adding 600 ppm urea, 0.124 SAPU/g ds FERMGEN™ 2.5× (an acid fungal protease), 0.33 GAU/g ds CS4 (a variant of Trichoderma reesei glucoamylase) and 1.46 SSCU/g ds Aspergillus kawachii α-amylase at pH 4.8.

50 grams of liquefact was weighted into 125 ml vessels and inoculated with fresh overnight cultures from colonies of the modified strains or FG strain at 32° C. and under the temperature ramp conditions. Samples were harvested by centrifugation at 55 hrs, filtered through 0.2 μm filters, and analyzed for ethanol, glucose, acetate and glycerol content by HPLC (Agilent Technologies 1200 series) using Bio-Rad Aminex HPX-87H columns at 55° C., with an isocratic flow rate of 0.6 ml/min in 0.01 N H₂SO₄ eluent. A 2.5 μl sample injection volume was used. Calibration standards used for quantification included known amounts of DP4+, DP3, DP2, DP1, glycerol and ethanol. The results of the analyses are shown in Table 9 and Table 10. Ethanol increase is reported with reference to the FG strain.

TABLE 9
Analysis of fermentation broth following fermentation
under temperature ramp conditions

| | Glucose | Ethanol | Ethanol |
|---|---|---|---|
| Strain | (g/L) | (g/L) | compared to FG |
| FG | 9.22 | 137.29 | 1 |
| YJL065c-DEL | 2.16 | 141.62 | 1.032 |
| EFB1-Dpb3 | 1.6 | 142.11 | 1.035 |
| EFB1-Dpb3- | 0.5 | 143.26 | 1.043 |
| YJL065c-DEL | | | |

As shown in Table 9, yeast harboring the YJL065c gene deletion in addition to the YBR278w gene controlled by EFB1 promoter (i.e., overexpressing Dpb3) produced significantly (i.e., almost 1%) more ethanol compared to yeast overexpressing Dpb3, alone, and over 4% more than the unmodified reference strain under temperature ramp conditions.

TABLE 10
Analysis of fermentation broth following fermentation at 32° C.

| | Glucose | Ethanol | Ethanol |
|---|---|---|---|
| Strain | (g/L) | (g/L) | compared to FG |
| FG | 0.63 | 141.33 | 1 |
| YJL065c-DEL | 1.85 | 142.51 | 1.008 |
| EFB1-DPB3 | 0.45 | 143.28 | 1.014 |
| EFB1-DPB3- | 0.54 | 144.07 | 1.019 |
| YJL065c-DEL | | | |

As shown in Table 10, yeast harboring the YJL065c deletion in addition to the YBR278w gene controlled by EFB1 promoter (i.e., overexpressing Dpb3) produced about 0.5% more ethanol compared to yeast overexpressing Dpb3, alone, and almost 2% more than the unmodified reference strain at 32° C.

What is claimed is:

1. Modified yeast cells derived from parental yeast cells, the modified cells comprising a genetic alteration that causes the modified cells to produce an increased amount of Dpb3 polypeptides compared to the parental cells, wherein the modified cells produce during fermentation an increased amount of alcohol compared to the amount of alcohol produced by the parental cells under identical fermentation conditions.

2. The modified cells of claim 1, wherein the genetic alteration comprises the introduction into the parental cells of a nucleic acid capable of directing the expression of a Dpb3 polypeptide to a level above that of the parental cell grown under equivalent conditions.

3. The modified cells of claim 1, wherein the genetic alteration comprises the introduction of an expression cassette for expressing a Dpb3 polypeptide.

4. The modified cells of claim 1, wherein the genetic alteration comprises the introduction of an exogenous YBR278w c gene.

5. The modified cells of claim 2, wherein the genetic alteration comprises the introduction of a stronger promoter in an endogenous YBR278w c gene.

6. The modified cells of any of claims 1-5, wherein the amount of increase in the expression of the Dpb3 polypeptide is at least about 30-fold compared to the level expression in the parental cells grown under equivalent conditions.

7. The modified cells of any of claims 1-5, wherein the amount of increase in the production of mRNA encoding the Dpb3 polypeptide is at least about 30-fold compared to the level in the parental cells grown under equivalent conditions.

8. The modified cells of any of claims 1-7, further comprising disruption of the YJL065c gene.

9. The modified cells of any of claims 1-8, wherein the cells produce a reduced amount of functional Dls1 polypeptides.

10. The modified cells of any of claims 1-9, wherein the cells do not produce Dls1 polypeptides.

11. The modified cells of any of claims 1-10, wherein the cells further comprise an exogenous gene encoding a carbohydrate processing enzyme.

12. The modified cells of any of claims 1-11, further comprising an alteration in the glycerol pathway and/or the acetyl-CoA pathway.

13. The modified cells of any of claims 1-12, further comprising an alternative pathway for making ethanol.

14. The modified cells of any of claims 1-13, wherein the cells are of a Saccharomyces spp.

15. A method for increasing the production of alcohol from yeast cells grown on a carbohydrate substrate, comprising: introducing into parental yeast cells a genetic alteration that increases the production of Dpb3 polypeptides compared to the amount produced in the parental cells.

16. The method of claim 15, wherein the cells having the introduced genetic alteration are the modified cells are the cells of any of claims 1-15.

17. The method of claim 15, wherein the cells having the introduced genetic alteration that increases the production of Dpb3 polypeptides further comprises a genetic alteration that reduces the amount of functional Dls1 polypeptides produced compared to the parental cells.
